(19) United States
(12) Patent Application Publication
(10) Pub. No.: US 2004/0243163 A1
(43) Pub. Date: Dec. 2, 2004

(54) Surgical instrument

(75) Inventors: Roy R. Casiano, Miami, FL (US); Phillip Andrew Ryan, Memphis, TN (US)

Correspondence Address: OLIFF & BERRIDGE, PLC, P.O. BOX 19928, ALEXANDRIA, VA, 22320, US

(73) Assignee: GYRUS ENT L.L.C, Bartlett, TN

(21) Appl. No.: 10/797,179

(22) Filed: Mar. 11, 2004

Related U.S. Application Data
(60) Provisional application No. 60/459,260, filed on Apr. 2, 2003.

(51) Int. Cl. A61B 17/32

(52) U.S. Cl. 606/170; 606/167

(57) ABSTRACT

A rotary tissue-cutting instrument forming a laryngeal blade (3) includes an outer tubular member (14) having a hub (4), a proximal portion (150) having a longitudinal axis and extending distally from said hub to a proximal bend (151) curving in a first direction. An intermediate portion (152) extends distally at an angle to the longitudinal axis of said proximal portion (150) in a first plane. The intermediate section extends from the proximal bend (151) to a distal bend (153) curving in a second direction, and a distal portion (154) extends distally from the distal bend (153) in a second plane. The distal portion (154) extends to a distal end having an opening (16) therein. An inner member (15) is rotatably disposed in the outer tubular member (14) and has a proximal end for mounting to a powered handpiece and a distal cutting tip (17) disposed adjacent the opening (16), the inner member being flexible adjacent said distal and proximal bends.

This invention relates to a surgical instrument such as a shaver or burr in which a surgical tool is rotatably driven by a handpiece. Such instruments typically include a rigid outer tube within which an inner tube is rotated, for example by a motor. A cutting implement, such as a cutting blade or abrading burr, is disposed on the distal end of the inner tube. Tissue or bone is exposed to the cutting implement through an opening in the distal end of the outer tube, and fragments cut by the cutting implement are drawn through the interior of the inner tube by the use of suction. Such instruments can employ tubes that are straight or curved, and U.S. Pat. No. 5,755,731 is a typical example of a device employing a curved tube.

U.S. Pat. No. 5,922,003 discloses a surgical tool having two curved regions. The outer tube has first and second curved regions, with a relatively long intermediate section therebetween. The tool is said to be suitable for laryngeal surgery, with the second bend curving in a direction opposite to that of the first bend, but all in a single plane.

The present invention attempts to provide an improvement to the laryngeal surgical tool of U.S. Pat. No. 5,922,003, which tool is more versatile and easier for the surgeon to use.

Accordingly there is provided a rotary tissue-cutting instrument forming a laryngeal blade comprising an outer tubular member having a hub, a proximal portion having a longitudinal axis and extending distally from said hub to a proximal bend curving in a first direction, an intermediate portion extending distally at an angle to said longitudinal axis of said proximal portion in a first plane, the intermediate section extending from said proximal bend to a distal bend curving in a second direction, and a distal portion extending distally from said distal bend in a second plane, the distal portion extending to a distal end having an opening therein, and an inner member rotatably disposed in said outer tubular member and having a proximal end for mounting to a powered handpiece and a distal cutting tip disposed adjacent said opening in said distal end of said outer tubular member, said inner member being flexible adjacent said distal and proximal bends.

The distal portion extending in a different plane to that of the remainder of the instrument allows surgeons to carry out precise laryngeal surgery, without needing to move their arm from a comfortable stable position. The device is compatible with the use of a supporting stand (such as a “Mayo” stand) on which surgeons rest their arm. The surgeon can navigate through the larynx without switching hands or needing to manoeuvre the instrument in awkward orientations.

In a preferred embodiment, the outer member is attached to the handpiece by means of a swivel collet, which allows the rotation of the outer member with respect to the handpiece. A swivel collet, such as that described in U.S. Pat. No. 5,492,527 and in our co-pending patent application U.S. Ser. No. 10/103104, allows the outer member and hence the cutting window to be easily rotated with respect to the instrument handpiece.

The first plane (containing the intermediate portion) and the second plane (containing the distal portion) are conveniently at an angle, one to another, of between 10 and 120 degrees. Preferably, the first and second planes are at an angle, one to another, of between 45 and 100 degrees, typically 60 to 90 degrees.

Preferably, the proximal portion has a length between said hub and said proximal bend, said intermediate portion has a length between said proximal bend and said distal bend, and said distal portion has a length between said distal bend and said distal end, said length of said intermediate portion being greater than said length of said proximal portion and being greater than said length of said distal portion. The proximal, intermediate and distal portions are conveniently each of straight configuration. According to a preferred embodiment of the invention, there is a suction passage extending along said inner member to permit aspiration of cut tissue.

The invention will now be described in greater detail, by way of example, with reference to the drawings, in which:

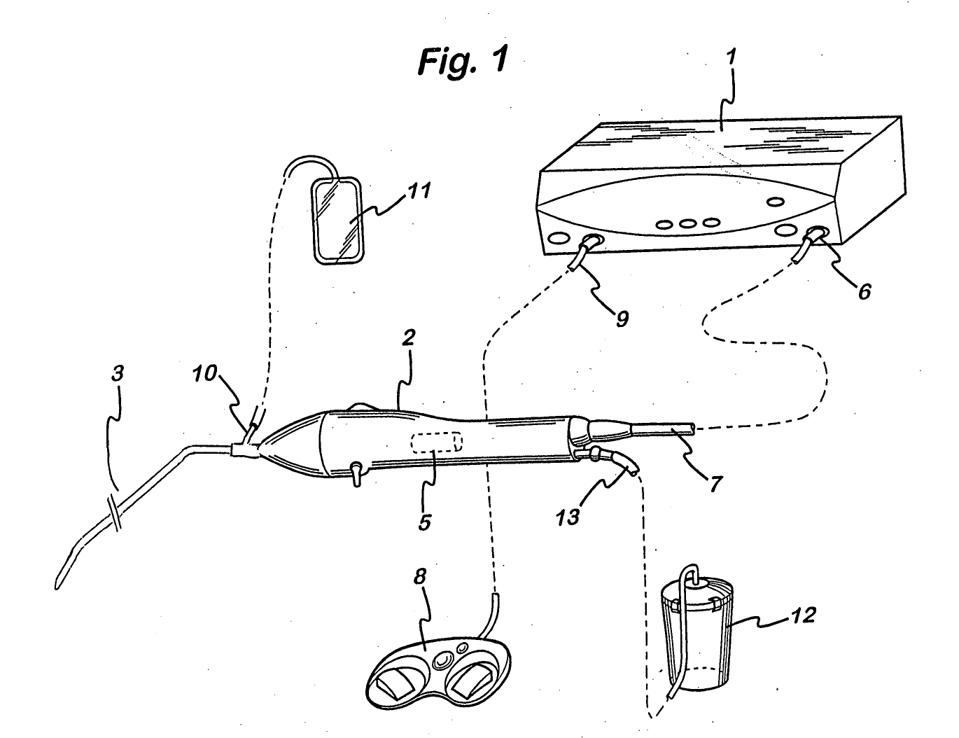

FIG. 1 is a schematic diagram of a surgical system incorporating a surgical instrument in accordance with the invention;

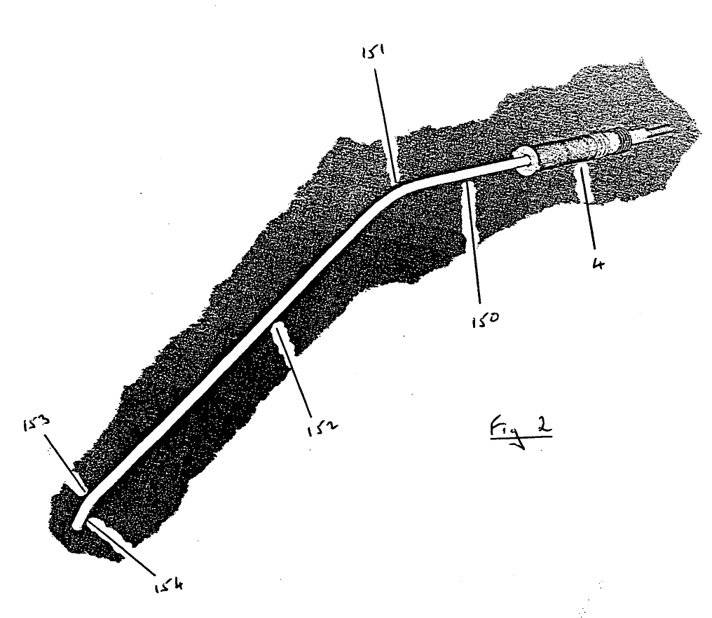

FIG. 2 is a side view of the blade of the surgical instrument of FIG. 1,

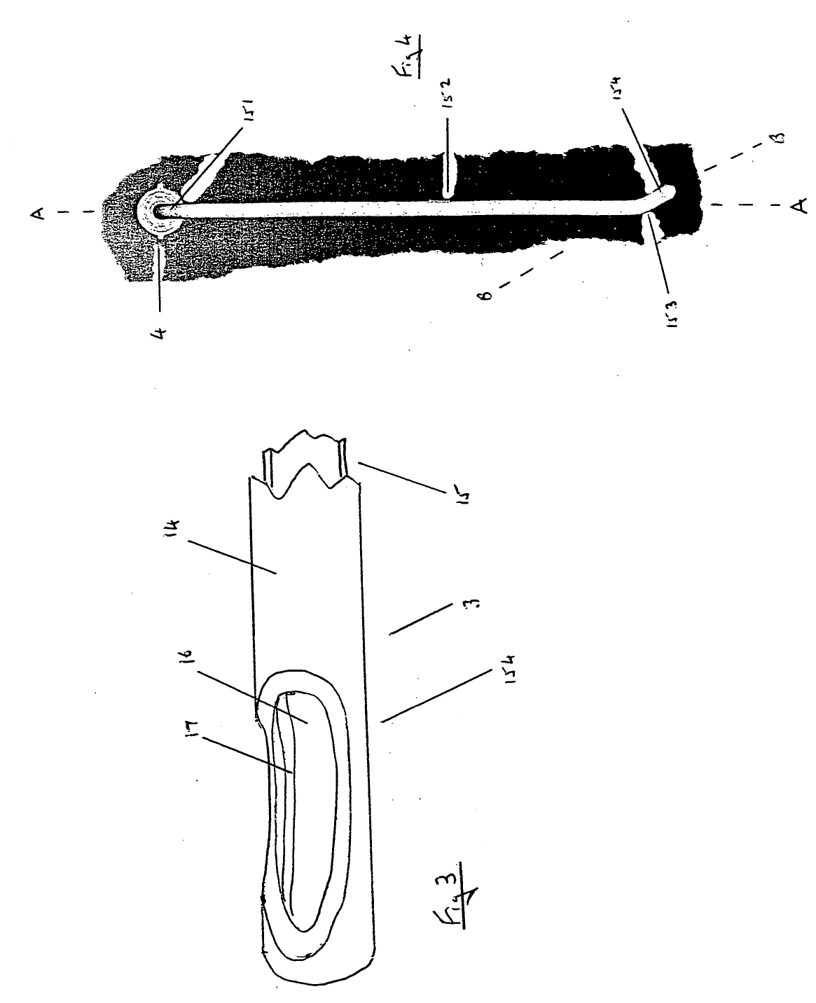

FIG. 3 is a side view, partly in section, of the distal end of the surgical instrument of FIG. 1;

FIG. 4 is an and view of the blade of FIG. 2;

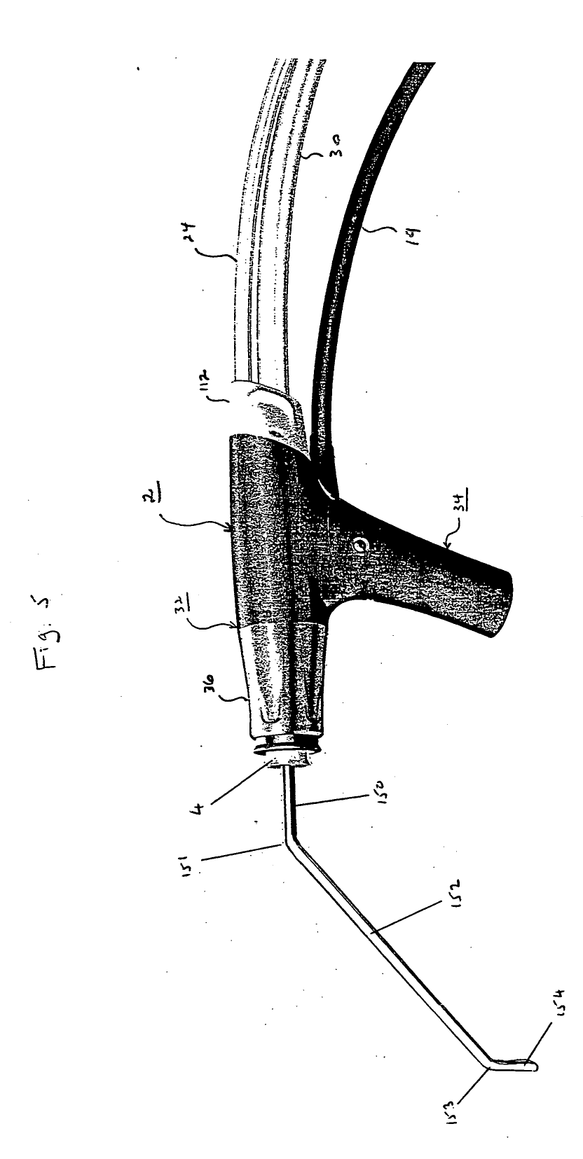

FIG. 5 is a side view of a surgical instrument in accordance with an alternative embodiment of the invention;

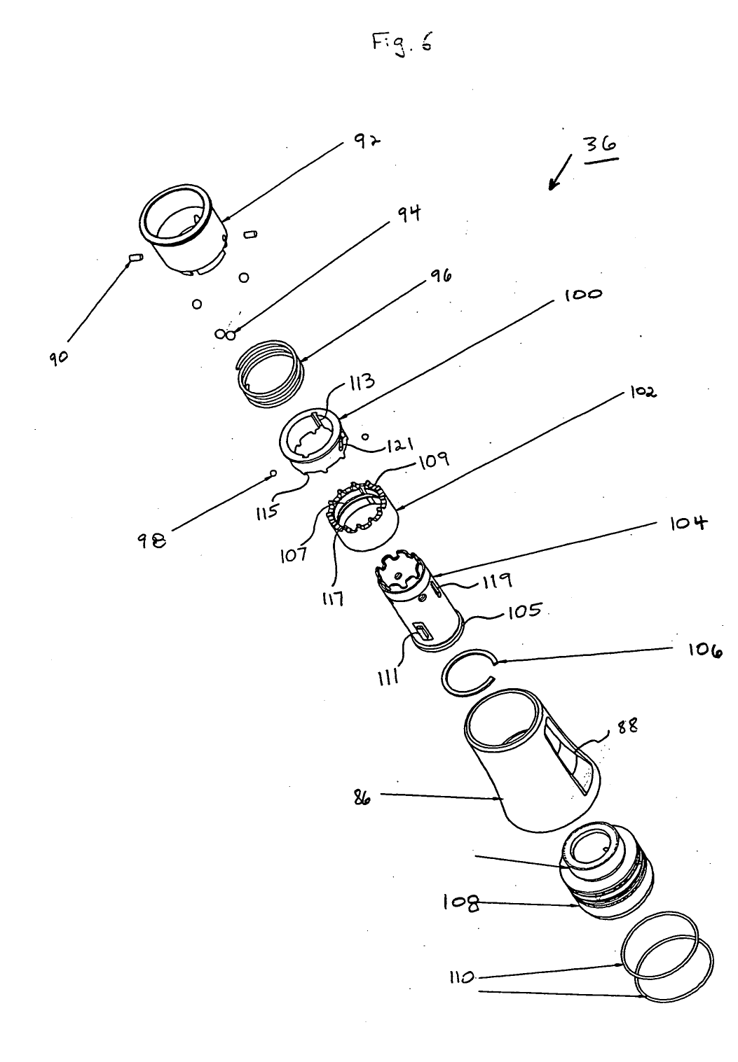

FIG. 6 is an exploded diagram showing the parts making up the collet assembly of the device of FIG. 5;

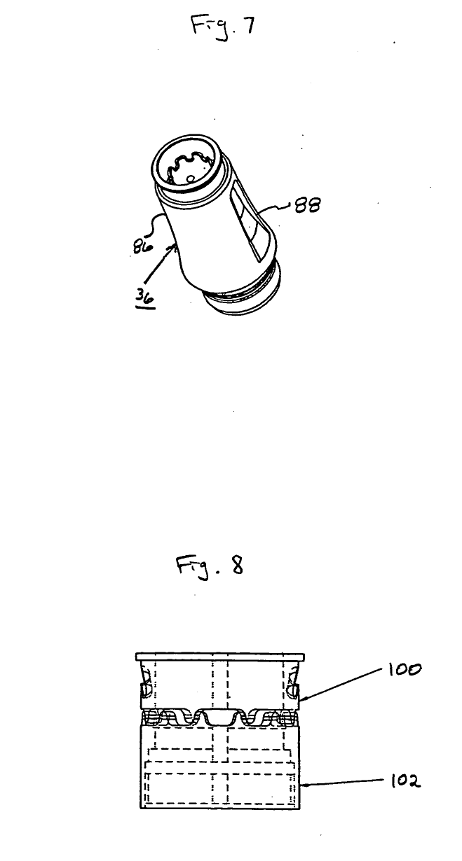

FIG. 7 is a perspective view of the swivel collet of FIG. 6; and

FIG. 8 is a side view, with hidden detail, of various parts of the swivel collet of FIG. 6.

Referring to the drawings, FIG. 1 shows a surgical system which includes a controller 1 and a handpiece 2 having a detachable surgical probe shown generally at 3. The probe 3 comprises an outer tubular member 14 and an inner tubular member 15. The inner member 15 is driven by a motor shown schematically at 5 within the handpiece. Power signals for the motor 5 are supplied to the handpiece 2 from an output socket 6 on the generator 1, via connector cord 7. Activation of the controller 1 may be performed by means of footswitch 8, coupled to the controller by means of connector cord 9. An inlet port 10 allows saline to be fed from a saline source 11 to the distal end of the probe 3. A source of suction 12 is also provided, coupled to the handpiece by cord 13.

Referring to FIGS. 2 and 3 the outer tube 14 of the probe 3 includes a hub 4, and a straight proximal portion 150 extending from the hub 4 to a proximal bend 151. The proximal bend 151 curves in a first direction such that a straight intermediate portion 152 extends from the proximal bend 151 at an angle of approximately 30 degrees to the proximal portion 150. The intermediate portion 152 extends from the proximal bend 151 to a distal bend 153. The distal bend 153 curves in a second direction such that a straight distal portion 154 extends at an angle of approximately 30 degrees to the intermediate portion 152. The distal portion 154 contains a cutting window 16.

The inner member 15 is contained within the outer tube 14 and driven for rotation by the motor 5. The inner member 15 is provided with flexible portions in the region of the proximal and distal bends 151 and 153. The inner member is also provided with a cutting tool 17 at its distal end, the cutting tool being accessible though the cutting window 16 in the outer tube 14.

FIG. 4 shows an end view of the probe 3. As can be seen from FIG. 4, the hub 4, proximal portion 150, and intermediate portion 152 are all in a first plane “A”. The distal bend 153 is such that the distal portion 154 is in a second plane “B”, at an angle of approximately 30 degrees to plane “A”. In use, the probe 3 is moved to engage tissue to be excised, and the tissue is drawn into the cutting window by the suction applied through the inner tubular member 15. When the tissue enters the cutting window 16, it is severed by the rotation of the cutting tool 17 and the excised tissue is evacuated by the suction along the inner member 15. The offset nature of the distal portion 154 allows laryngeal surgery to be carried out with minimal requirement for repositioning of the instrument.

FIG. 5 shows an alternative embodiment of surgical device in which the handpiece 2 includes an upper portion 32 and a lower portion 34 defining a pistol grip arrangement. The upper portion 32 extends generally parallel to the probe 3, while the lower portion 34 extends at an angle thereto. The probe 3 is attached to the upper portion of the handpiece 2 by means of a collet assembly 36. The motor 5 (not shown in FIG. 4) is located in the lower portion 34 of the handpiece, and is controlled by signals via control line 19. Fluid irrigation and suction are provided to the handpiece 2 via dual tubing 31, the fluid supply being via tube 24 and the suction supply via tube 30. The dual tubing 31 is attached to the handpiece 2 by means of a connector 112.

The swivel collet assembly 36 is shown in more detail in FIGS. 6 to 8. As shown in FIG. 5, the collet assembly 36 is provided at the front end of the upper portion 32 of the handle 2. Disposing the collet assembly 36 at this location enables an operator, such as a surgeon, holding the handle 2 in a pistol grip manner, to touch and rotate the assembly collet 36 or a portion thereof with the tip of at least one of the surgeon's fingers. Rotating at least a portion of the collet assembly 36 in this manner enables the cutting window of the probe 3 to rotate, thereby orienting the direction of the shaving and/or cutting of the desired bodily material.

As shown in FIGS. 6 and 7, the collet assembly 36 includes a swivel shell 86 that defines at least one gripping channel 88. The at least one gripping channel 88 enhances the surgeon's ability to grip the collet assembly 36 with the tip of at least one of the surgeon's fingers so as to rotate at least a part of the collet assembly 36. FIGS. 6 and 8 show a combination of sub-elements that enable manual rotation of the swivel shell 86 to change the orientation of the cutting window while the inner blade of the probe 3 rotates. The collet assembly includes release pins 90, a release ring 92, retention balls 94, a lock spring 96, unlocking balls 98, a sliding cam 100, a stationary cam 102, a retention sleeve 104, a retaining clip 106, the swivel shell 86, a base mount 108, and base mount seals 110.

The collet assembly includes a stationary cam 102 which is attached to the base mount 108 such that an interior gap defines a location for the retention of a flange 105 on the proximal end of the retention sleeve 104, thus capturing the retention sleeve and preventing it from moving axially, but allowing it to rotate freely and concentrically with respect to the main axis of the collet assembly. One method of capturing the flange on the retention sleeve is to use a retaining clip 106 which fits into an internal groove 107 in the stationary cam and defines a gap which ensures that rotation is free, but that axial movement is restricted. The use of the retaining clip further facilitates the assembly of the mechanism, by allowing the base mount 108 to be assembled into contact with the retaining clip 106 thereby setting the relative position of the base mount to the stationary cam and eliminating the need to adjust this engagement by manual means.

Two interior grooves 109 are located on the stationary cam 102 to provide relief to allow the cam to slide over two keys 111 on the exterior of the retention sleeve. These two grooves are provided as a means to aid assembly and are not functional once the collet assembly 36 has been completed. The sliding cam 100 also has two interior grooves 113 which engage with the keys 111 on the exterior of the retention sleeve 104 preventing relative rotational motion of these parts, but allowing the sliding cam 100 to slide freely in an axial direction along the length of the retention sleeve 104. This engagement is the means by which rotational motion is transmitted between the sliding cam 100 and the retention sleeve 104 and subsequently to the blade hub when the swivel shell 86 is rotated. The sliding cam engages 100 with the stationary cam 102 by means of teeth 115 and 117 that are located on the faces of each part facing towards each other. The teeth 115 and 117 are held in engagement by the spring 96 which is in turn retained by the release ring 92 which is retained by the release pins 90 which are engaged in holes in the release ring 92 and whose ends are placed in slots 119 in the retention sleeve 104. The release pins 90 are retained by the assembly of the swivel shell 86 which prevents the pins from falling out the holes which capture them in the release ring 92.

The teeth 115 and 117 on the cams 100 and 102 that engage with each other have geometry which when urged into engagement by the lock spring 96, are not permitted to slide against each other by means of friction. In order to prevent sliding of the teeth against other the contact angle of the teeth is substantially less than 45 degrees and in this case is 15 degrees. The grooves 121 on the exterior of the sliding cam 100 are shaped with a V profile and receive the unlocking balls 98 which engage in pockets inside the swivel shell 86. The balls 98 slide in the V shaped grooves 121 in the sliding cam 100 when the swivel shell 86 is rotated. Rotation of the swivel shell 86 by the surgeon causes a corresponding rotation of the sliding cam 100, lifting the sliding cam 100 out of engagement with the stationary cam 102. Once the sliding cam is free from the stationary cam, it can cause a corresponding rotation of the retention sleeve 104. In this way, a rotation of the swivel shell 86 causes a reorientation of the cutting window in the probe 3, via retention sleeve 104. However, should the retention sleeve be urged to rotate, for example by the probe 3, the rotation will be prevented by the engagement of the sliding cam 100 in the stationary cam 102. The action of the swivel shell 86 to lift the sliding cam 100 out of engagement with the stationary cam 102 means that while a rotation of the swivel shell will cause a corresponding rotation of the retention sleeve 104, the reverse will not be permitted (i.e. an attempt to rotate the retention sleeve 104 will not cause a corresponding rotation of the swivel shell 86). This provides the assurance that in the event of a jam the swivel shell will be prevented from rotating, thereby avoiding the possibility of injury to the surgeon.

As will be seen from the above, the surgeon can easily rotate the probe 3 by rotating the swivel shell 86. Thus the orientation of the cutting window 16 with respect to the handpiece 2 can vary during a surgical procedure. The offset nature of the distal portion 154 of the probe 3 enables the surgeon to access tissue in the larynx without excessive or awkward movement of the instrument.

1. A rotary tissue-cutting instrument forming a laryngeal blade comprising an outer tubular member having a hub, a proximal portion having a longitudinal axis and extending distally from said hub to a proximal bend curving in a first direction, an intermediate portion extending distally at an angle to said longitudinal axis of said proximal portion in a first plane, the intermediate section extending from said proximal bend to a distal bend curving in a second direction, and a distal portion extending distally from said distal bend in a second plane, the distal portion extending to a distal end having an opening therein, and
an inner member rotatably disposed in said outer tubular member and having a proximal end for mounting to a powered handpiece and a distal cutting tip disposed adjacent said opening in said distal end of said outer tubular member, said inner member being flexible adjacent said distal and proximal bends.

2. An instrument according to claim 1 wherein the first and second planes are at an angle, one to another, of between 10 and 120 degrees.

3. An instrument according to claim 2 wherein the first and second planes are at an angle, one to another, of between 45 and 100 degrees.

4. An instrument according to claim 3 wherein the first and second planes are at an angle, one to another, of between 60 and 90 degrees.

5. An instrument according to claim 1 wherein the proximal, intermediate and distal portions are each of straight configuration.

6. An instrument according to claim 1 wherein said proximal portion has a length between said hub and said proximal bend, said intermediate portion has a length between said proximal bend and said distal bend, and said distal portion has a length between said distal bend and said distal end, said length of said intermediate portion being greater than said length of said proximal portion and being greater than said length of said distal portion.

7. An instrument according to claim 1 wherein there is a suction passage extending along said inner member to permit aspiration of cut tissue.
